(19) United States
(12) Patent Application Publication
(10) Pub. No.: US 2010/0191195 A1
(43) Pub. Date: Jul. 29, 2010

(54) CANNULATED APPARATUS AND METHOD RELATING TO MICROFRACTURE AND REVASCULARIZATION METHODOLOGIES

(76) Inventor: Ira Kirschenbaum, Scarsdale, NY (US)

Correspondence Address: Ira Kirschenbaum, 82 Brookby Road, Scarsdale, NY, 10583, US

(21) Appl. No.: 12/377,437

(22) PCT Filed: Oct. 9, 2007
(86) PCT No.: PCT/US07/80868

§ 371 Date: Apr. 1, 2010

Related U.S. Application Data
(60) Provisional application No. 60/828,654, filed on Oct. 8, 2006.

(51) Int. Cl. A61M 5/32

(52) U.S. Cl. 604/272

(57) ABSTRACT

The present invention relates to a carrnulated microfracture kit, apparatus, and method for using the same during a medical treatment. The present kit enables precise and repeated positioning, the regulation and repetition of microfracture force application, and a control of a mosaic bone penetration and other surgical control improvement features. The present invention overcomes the detriments resultant from prior techniques in an apparatus that is readily adaptable to a variety of adaptive orthopedic surgical procedures. Assembled and selectable kits are provided.

### CROSS REFERENCE TO RELATED APPLICATION

This application claims priority to U.S. Provisional Application Ser. No. 60/828,654 filed Oct. 8, 2006, the entire contents of which are herein incorporated by reference.

### BACKGROUND OF THE INVENTION

1. Field of the Invention

The present invention relates to a cannulated microfracture apparatus and methods for implementing the same. More specifically, the present invention relates to a cannulated delivery apparatus functionally employing a microfracture device and a method for operating the same to augment revascularization.

2. Description of the Related Art

Within the broad field of orthopeadic (orthopedic) surgery, various physical techniques and methods have been developed to aid revascularization of arthritic or otherwise damaged or necrotic bone; principally in localities proximate knee, hip, and ankle joints, although there is no limitation to these revascularization regions.

Previously employed methods included (a) high speed burrs (debridement), (b) sole-use smooth pin members, (c) sole-use microfracture picks, (d) subchondrial drilling, and other methods commonly supported with additional anthroscopic lavage and other processes to rid a joint of resultant loose debris. Each methodology has characteristics now recognized by those of skill in the art as negatives to beneficial patient outcome thereby providing a need for the present invention.

The employment of debridement burrs, smooth pins, and drilling has fallen out of favor due to the consequential heat necrosis or cell death brought on by in situ heat buildup.

Due to this difficulty and others, the current favored technique for microfracture employs the use of hand-held and hand-guided picks formed of a solid member with a pointed end. During use, a surgeon places the pick tip through an anthroscopic portal and applies (or attempts to provide) suitably-directed percussive pressure to the end point by simply hitting the back of the pick with a mallet, hammer, or their hand. Unfortunately, due to simple human error the resultant force drives the generally conical tip into the target bone at an angle other than axial to the point itself often damaging the bone and the preferably-reached subchrondral bone, forming one or a plurality of non-uniform holes. Such holes are generally arrayed in an undesirably interfering and irregular or overlapping mosaic fashion based upon the inaccuracy of physical-directed positioning (leading to improper angle, penetration depth, and force use errors).

In contrast to say drilling, microfracture has substantial advantages beyond the avoidance of heat build up. In addition to the lack of heat necrosis, the pick-tip creates an increased surface area for clot formation while allowing a general structural integrality to remain in the subchrondrial bone. For a broader review of revascularization techniques reference is made to “New Techniques for Cartilage Repair and Replacement by Stone, et al, http://www.stoneclinic.com (visited Jun. 7, 2008), the entire contents of which are herein incorporated by reference.

It is also to be understood that the existence of conventional laproscopic cannulas are known in the art from U.S. Pat. No. 4,112,932 the entire contents of which are herein incorporated fully by reference.

Unfortunately, a number of detriments have not been appreciated by the prior art, namely resulting from employing the current microfracture pick techniques. These detriments include:
(a) The further a user drives the pick into the bone uncontrollably resulting in a wider-than-optimal part of the resultant hole.
(b) Substantially all of the pick angles at the end of each pick-tool shaft do not drive well or cleanly (meaning linearly to a pick-tip-axis with the application of force along a pick-tip axis).
(c) The surgeon is often unable to apply sufficient, regular, or uniform force to drive the pick consistently deeply enough to effect a desired medical outcome—resulting in unsuccessful re-vascularation locations.
(d) A surgeon is unable to reliably and repeatedly reposition the pick relative to the desired bone target following successive uses, thereby resulting in scattered, inaccurate and potentially damaging hole placement and a general difficulty in creating a uniform mosaic pattern for revascularization.

Accordingly, there is a need for an improved cannulated apparatus and method relating to microfracture and revascularization methodologies.

### SUMMARY OF THE INVENTION

An aspect of the present invention is to provide an apparatus and method to overcome at least one of the detriments noted above.

Another aspect of the present invention is to provide a cannulated microfracture kit that allows a regulated application of microfracture force and comprehensive control of microfracture location.

Another aspect of the present invention is to provide a microfracture kit providing a surgical user with customizable microfracture options readily adapted to a particular skeletal or joint geography and structure.

Another aspect of the present invention enables a user to finely regulate and controllably vary a microfracture penetration depth, e.g. depth control or penetration control system or means allowing adjustment and control of depth penetration.

Another aspect of the present invention is to provide a universal microfracture system that readily adapts to personal-use differences in surgical striking techniques.

Another aspect of the present invention is to provide a positioning and repositioning system and means that readily adapts to alternative angles without varying a depth of penetration.

Another aspect of the present invention is to provide a non-driving positioning system, allowing a positive positioning point proximate to microfracture location, wherein controlled microfracture into the microfracture location by a trocar does not drive the positioning system, allowing use of more precise and diversely adaptable positioning systems that do not co-operate as a microfracture driving point, e.g. non-driving positioning system.

Another aspect of the present invention is to provide a non-driving positioning cannulated system slidably separable from a microfracture trocar member, wherein a microfracture driving force driven along the axis of the microfracture trocar does not impact the positioning cannulated system positioning point.

Another aspect of the present invention is to provide a cannulated microfracture kit that is readily arranged as a pre-packaged system for convenient surgical use, and may optionally allow broken-down kits allowing ready selection by a user of a number of diverse assembly options during a use.

The present invention relates to a cannulated microfracture kit, apparatus, and method for using the same during a medical treatment. The present kit enables precise and repeated positioning, the regulation and repetition of microfracture force application, and a control of a mosaic bone penetration array. The present invention overcomes the detriments resultant from prior techniques in an apparatus that is readily adaptable to a variety of surgical procedures to speed patient recovery.

According to an embodiment of the present invention there is provided a cannulated device having an optional rigid or flexible curved cannulated transfer assembly that enables steady positioning proximate a target surface.

Another aspect of the present invention is to provide an embodiment wherein a pick end is optionally pointed, employs a helical geometry enabling a threaded bone engagement or simple additional bone penetration, or employs a series of force-regulation rings enabling repeated force use.

According to another aspect of the present invention, there is provided a microfracture hand tool having a striking surface distal a pick end member.

According to another aspect of the present invention, there is provided a kit including a depth-of-penetration-stop mechanism enabling a user-surgeon to regulate pick-penetration within a force range, thereby improving a regulation of penetration during use.

The above, and other aspects, features and advantages of the present invention will become apparent from the following description read in conduction with the accompanying drawings, in which like reference numerals designate the same elements.

### BRIEF DESCRIPTION OF THE DRAWINGS

FIG. 1A is a perspective view of an external articulated joint locating a cannulated microfracture apparatus according to one aspect of the present invention noting a rotational depth control system and off-set co-axial axis set point.

FIG. 1B is an exploded view of one aspect of a cannulated microfacture apparatus according to the present invention.

FIG. 1C is an exploded partial assembled view of a driving handle of a striker assembly.

FIG. 1D is a first positioning view of a cannula pick end penetrating the patient's skin pocket prior to a striking motion.

FIG. 1E is an operative perspective view noting the ease of positioning the axis set depth and angle control relative to a pre-placement positioning.

FIG. 1F is an operative perspective view of a pick and cannula having a cone end as in FIG. 1B.

FIG. 1G illustrates adaptive application of angles employing the present positioning system and apparatus (these are noted in exemplary displays in FIGS. 1I-1L).

FIG. 1H is a perspective view of an exemplary cannula member relative to a first set point position demonstrating the accuracy of multiple angularized repositioning employing the present set point system.

FIGS. 1I-1L depict representative mosaics of regularized microfracture penetrations in bone pivoted about a cannula pick tip end, and noting the ability to controllably position microfracture locations in a plurality of non-overlapping, and intentionally overlapping patterns depending upon a required therapeutic determination by a medical professional.

FIG. 1M is a first optional striker end of a trocar as viewed in region I in FIG. 1A according to the present invention.

FIG. 1N is a second optional striker end of a trocar viewed in region II in FIG. 1B according to the present invention.

FIG. 1O is a third optional striker end of a trocar according to the present invention.

FIG. 1P is a fourth optional striker end of a trocar according to the present invention.

FIG. 1Q is a fifth optional grinding/cutting/cleaning end of a trocar according to the present invention.

FIG. 2A is an exploded view of one aspect of an alternative cannulated microfracture apparatus according to another aspect of the present invention.

FIG. 2B is a partial close up view along orientation of section 1I-1I and view III in FIG. 1G prior to microfracture and employing the construction of portion V in FIG. 2A.

FIG. 2C is a partial close up view along section 1I-1I in FIG. 1G (as shown in FIG. 2B) upon initial microfracture.

FIG. 2D is a close-up view of the alternative threaded trocar tip end in FIG. 2C.

FIG. 2E is a re-positioned close up view of a microfracture system according to FIG. 2B, having an angularized displacement along angle A based on a first co-axial set point position, and noting an alternative angle tip construction.

FIG. 2F is a re-positioned close up view of a microfracture system placement as in FIG. 2E, noting an alternative angle with the same set point position.

FIG. 2G is a first type of axis set or tip end of a cannula member represented in position in IV in FIG. 1B.

FIG. 2H is a second type of axis set or tip end of a cannula member.

FIG. 2I is a third type of axis set or tip end of a cannula member.

FIG. 2J is a fourth type of axis set or tip end of a cannula member having a depth-penetration stopping construction.

FIG. 2K is a fifth type of axis set or tip end of a cannula member having a multi-step or multi-force depth-penetration stopping construction.

FIG. 2L is a sixth type of axis set or tip end of a cannula member having a replaceable and extending replaceable tip-end member.

FIG. 2M is a seventh perspective exploded view of a cannulated microfracture apparatus according to another aspect of the present invention having two set pick points on a replaceable and pivotable pick end member.

FIG. 2N is an eighth perspective exploded axis set having a dual-point or multi-point replaceable end for threadable-assembly prior to entering a skin opening.

FIG. 3A is a ninth perspective view of an axis set or tip end having a smooth annular tip portion and a variable adjustment member, enabling variable positioning by a surgical user by bending and adaptively-positioning the pick-point prior to or after a skin-penetration for enhances surgical freedom.

FIG. 4A is a perspective exploded view of a cannulated microfracture apparatus according to another aspect of the present invention.

FIG. 4B is a close up view of portion VI in FIG. 4A noting an adaptive threaded end of a cannulated trocar device for bone-threading penetration and enhanced security, resistance to unintended displacement, and enhanced bone micro-fracture surface area.

FIG. 4C is a partial sectional penetrative view of the apparatus of FIG. 2A penetrating bone.

FIG. 4D is a partial penetrative view of the apparatus of FIG. 2A penetrating bone following the action of FIG. 2C.

FIG. 5A is a cannulated microfracture kit for rapid use assembly view noting selectably-contained kit elements of varying types and lengths.

FIG. 5B is a close up view of a first threaded trocar end in view IX in FIG. 5A.

FIG. 5C is a close up view of a threaded trocar end in view X in FIG. 5A having a larger diameter than FIG. 5B.

FIG. 5D is a larger close up view of a threaded trocar end in view XI in FIG. 5A having a larger diameter than FIG. 5C.

FIG. 5E is a side view of a first threaded pilot hole produced in a bone member, using for example the threaded trocar in view IX in FIG. 5B.

FIG. 5F is a side view of the formation of a larger threaded hole employing a first-produced threaded hole in FIG. 5E as a guide and larger diameter trocar from view XI in FIG. 5A.

FIG. 6A is another cannulated microfracture kit assembly view noting alternative contained elements.

FIG. 6B is a trocar tip end in view XII of FIG. 6A for inserting a biological aid such as a growth media, a growth media containing membrane, or cement on a designated bone site.

FIG. 6C is a cross sectional view of FIG. 6B along line 6C-6C.

FIG. 6D is an alternative multi-micro pin trocar tip end aspect of the present invention having multiple pick points.

FIG. 6E is an alternative trocar shall construction as seen in view XIII in FIG. 6A.

FIG. 6F is an alternative spiral trocar shaft construction as seen in view XIV in FIG. 6A having a common outer diameter

FIG. 6G is an alternative spiral trocar shaft construction a seen in view XV in FIG. 6A having an external thread spiral to aid debris removal.

FIG. 7A is a cannulated microfracture kit assembly view noting an alternative aspect of the present invention supporting a multi-strike function without repositioning employing multi-strike trocars.

FIG. 7B is a cross sectional view along line 7C-7C in FIG. 7A.

FIG. 7C is a cross sectional view along line 7D-7D in FIG. 7A.

FIG. 7D is a close up view of an operative end of a single trocar and multi-opening cannula in FIG. 7A during use with a fluid flow noting impact.

FIG. 7E is a close up view of an operative end of a dual trocar use from the inventive aspect of FIG. 7A showing the second trocar penetration use during continued fluid flow.

FIG. 8A an exploded cannulated microfracture kit assembly view noting alternative guide construction features according to another aspect of the present invention where the guiding slide is external.

FIG. 8B is an assembled view of the kit assembly of FIG. 8A.

FIG. 8C is a cross sectional view along line 8C-8C in FIG. 8B.

FIG. 8D is a close up view of the assembly in FIG. 8A positioned for a first microfracture use.

FIG. 8E is another exploded view of a cannulated microfracture kit assembly similar to that in FIG. 8A, having multi-trocar adaptation and an alternative guide assembly with rounded penetration snout.

FIG. 8F is an assembled view of the kit assembling of FIG. 8F.

FIG. 8G is a cross sectional view along line 8G-8G in FIG. 8F.

FIG. 8H is a close up view of the assembly in FIG. 8E positioned for a first multi-microfracture use.

FIG. 9A is an optional microfracture kit assembly containing selectable length items and variable striking and support heads.

### DETAILED DESCRIPTION OF THE PREFERRED EMBODIMENTS

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, and below may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

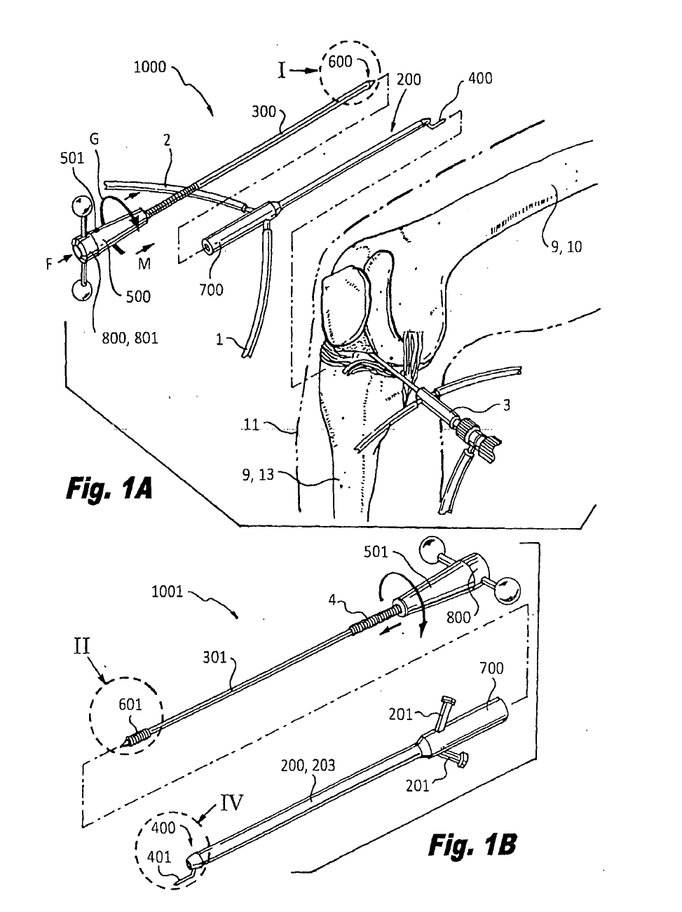

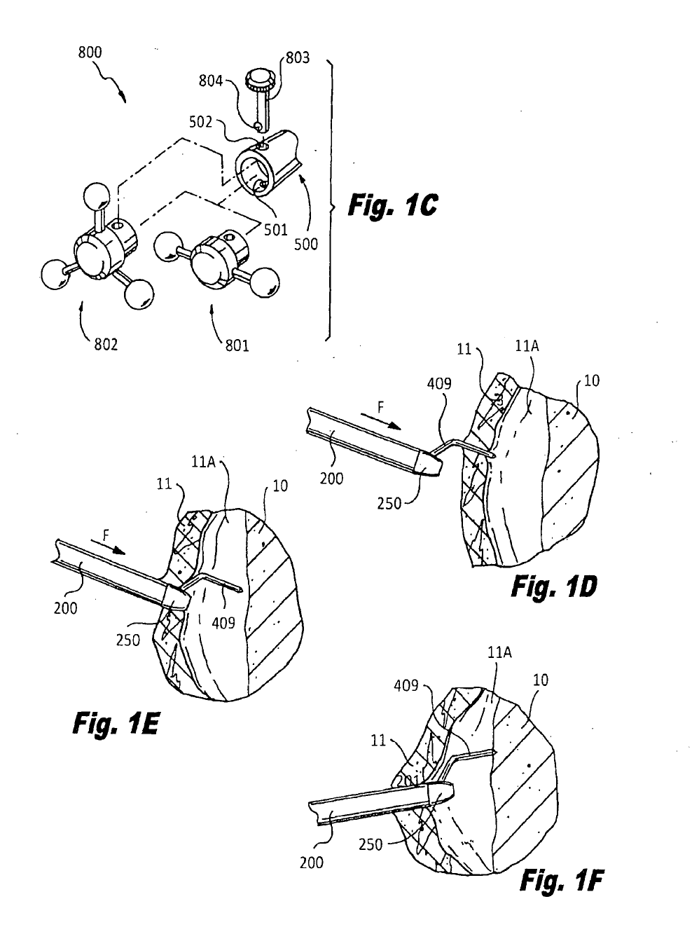

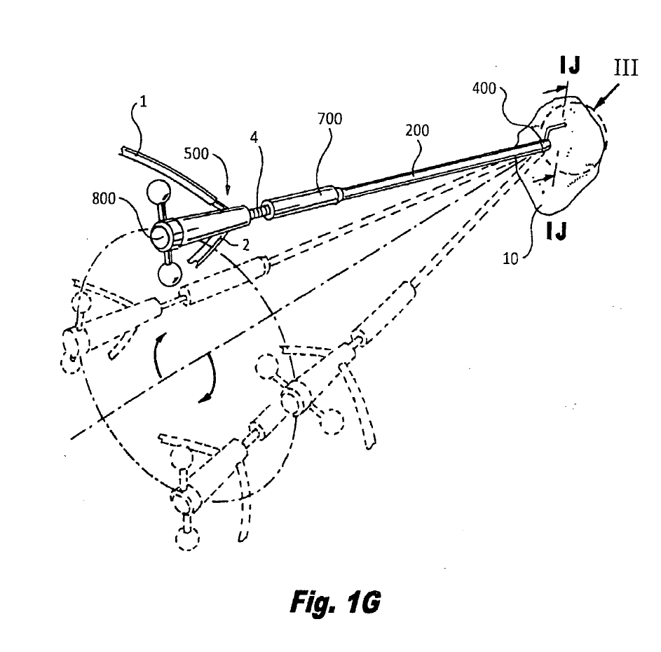

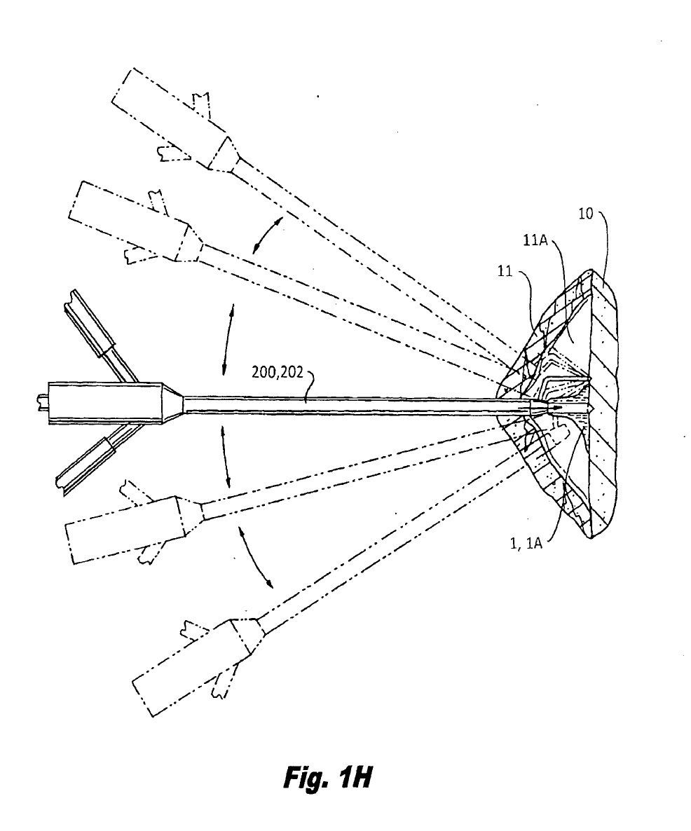

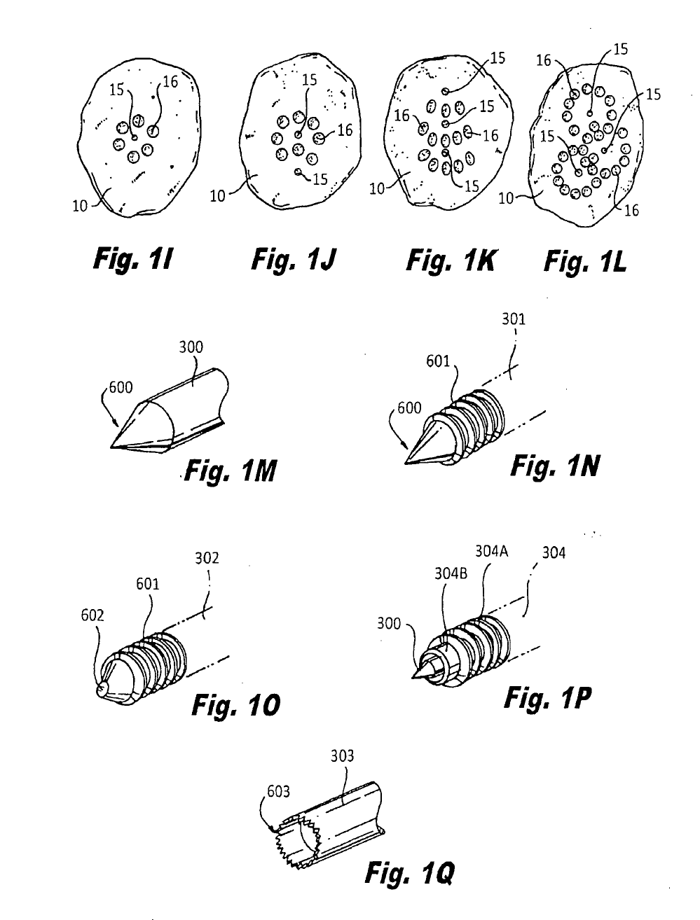

Referring now to FIGS. 1A-1C, a first and second cannulated microfracture system 1000, 1001 each include respectively a trocar 300, 301 having respective contact tip ends 600, 601 on proximal ends thereof (shown here as preferably co-axially located but this is not required), a threadably adjustable striker system 500 having a threadably-adjustable striker member 501 on a distal end thereof and a handle member or grip/striking assembly 800.

Strikers 500 include a striker or striking end thereof 501, a receiving hole 502 (FIG. 1C) for an optional pin member 803 having a securing detent ball 804, as shown for accepting a striker grip assembly 800 having optional T-handle ends 801 and Tri-handle ends 802 to aid hand control and twisting or shifting during use.

A cannula 200, 203 includes an axis set or pick end 400 on a proximal end and a sheath end 700 on a distal end thereof. Pick end member 400 includes, in this embodiment, and a single pick point 401. Sheath end 700 includes optional ports 201, 201 adaptive to threadably accept a Luer Lock Tip device (an example is produced by Terumo Medical Corp, Elkton, Md. 21921) (not shown), or optionally a fluid flow member 1 and vacuum member 2, or any other common surgical supply/suction or aid system used conventionally during orthopedic surgery. A representative physical joint is shown to aid contextural understanding, here employing a knee joint of a leg 9 having hinged bones 10, 13 and imaged with a conventional imaging or scope unit 3 penetrating a flesh or skin layer 11.

Focusing now on alternative microfracture system 1001 and trocar 301 in FIG. 1B (but also shown in FIG. 1A) a threaded region 4 optionally surrounds an outer region proximate striker or striking end 500. It is to be understood, that striker 500 is threadably mounted on threaded region 4 of trocar 301 (and 300), and is adjustable lengthwise along the trocar's axial length by rotation in either direction G, a surgically desirable distance M, so as to adjust a penetration distance resultant from an applied force F, upon contact with sheath end 700, as will be discussed.

During use a contact end of striker 500 impacts an impact region of each respective sheath end 700 which, being pre-positioned by a user on the bone, stops the forward motion of the 300 trocar and hence penetration of the bone at a desired depth. As a consequence, it will be recognized that the present system provides one form of adjustable and selectable depth adjustment means for controlling and pre-determining a microfracture depth for patient safety by simply user-adjusting the length of the trocars 300, 301 received within striker 500 via threadable adjustment. As a consequence, those of skill in the art will recognize that an effective length of a trocar, measured between striking end 501 and tip ends 600, 601 may be readily adjusted by manual manipulation.

During an operative use, it is envisioned that a surgeon will position systems 1000, 1001 in contact with a bone requiring microfracture treatment and will initially strike either striker end 501 of striker 500 or, upon assembly with kit 800, the rear end of handle assembly 800 depending upon personal choice. In either assembly condition, force F is transmitted axially along a length of trocars 300, 301 to tips 600, 601 for contacting bone.

As a benefit of the present depth control or depth adjustment system being connected with the respective trocars, it will be recognized that the depth adjustment system will additionally operate when striker 500 is assembled with handle assembly 800 thereby providing a user maximum freedom of choice in a fast-paced surgical environment. As an optional technique, following initial operation of axial Force F, a user may grip handle assembly 800 for simple removal, rotation (clock-wise or counter clockwise), prying, repositioning, or otherwise manipulate systems 1000, 1001 in a substantially inelastic manner to achieve a beneficial surgical result.

Referring now to FIGS. 1D through 1F, a possible series of method steps for the present systems is provided. As noted in FIG. 1D, a cannula 202 is provided with a sheath end 700 (not shown) on a proximate end and an adaptive pick point or axis set 409 as discussed above.

In FIG. 1E, a cut in a skin layer 11 allows a surgical approach to bone surface 10. As shown, pick point 409 on canula 202 first enters the cut and is urged through skin layer 11 creating a small skin pocket 11A above the surface of bone 10. As is shown, smooth bullet end 250 is adaptively used to on end 400 so as to ease entry through elastomeric skin 11 to allow cannula 200 to position tip end 409 on bone 10. As should be recognized from the images, skin is elastomeric and provides a sealing contact with the external surface of cannula 202.

Referring now specifically to FIGS. 1E and 1F, it is readily apparent that cannula 202 may be easily positioned, and repositioned reliably relative to the bone-pick point connection within open region 11A, so as to prohibit cannula 202 from slipping relative to bone 10 prior to a microfracture use. A particular advantage, but not a requirement, of this construction, is that a user may rapidly reposition cannula 202 (as will be discussed) for causing microfractures without having to guess at a location, because a pick-point location is a controlled reference position. The bone-pick point connection with 409 remains as a secure and intentionally-movable reference point that allows a surgeon to rapidly create the microfractures required for revascularization.

Referring now to FIGS. 1G and 1H, it is envisioned that either microfracture system 1000, 1001 may be positioned on a respective axis CL set or prick point apparatus 400 on an outer surface of a bone member 10, and rotated or pivoted as desired about a plurality of angles (as shown) in order to enable positioning highly-accurate microfractures to promote bone health and biological revascularization in a manner noted in the orthopedic literature by those of skill in the art.

As will be similarly appreciated in FIG. 1H, angularized motion along a common plane may also be achieved relative to the pick-point position on bone 10, while either conducting microfracture treatment or when flushing with a fluid 1, 1A, as shown.

Referring now to FIGS. 1I through 1L, a surface of bone 10 is noted centered on an initial pick point location 15, or on a plurality of initial pick point locations 15, as shown. Employing the process noted in FIGS. 1G, 1H, as well as lift-and-reposition methods, it should be readily apparent to those of skill in the surgical arts, that regularly spaced microfracture locations 16 may be uniformly spaced at a common depth by employing the present system, and may similarly be placed at related positions by simply manipulating, rotating, and tipping, cannula 200 relative to an initial insertion direction about point 15. In this way it must be appreciated that a plurality of depth-controlled and position-controlled microfractures may be created on a bone surface, allowing ready avoidance of diseased or damaged bone. For example, as show in FIG. 1I, a simple ring may be created, or as in FIG. 1J, a single or series of arcs may be created, or optionally combinations thereof without departing from the scope and spirit of the present invention.

Referring now to FIGS. 1M-1Q, a plurality of alternative trocar tip ends and constructions are provided. As noted earlier, a smooth trocar 300 may include, for example a single trocar smooth end 600, and may obviously include differing trocar diameters (for example 1.2 mm, 2.0 mm, 2.5 mm, 3.0 mm and upwardly to, any user desired diameter) without departing from the teachings herein. It is envisioned that for a common fixed trocar similar to 300 a minimum diameter will be approximately 18 gauge for structural strength reasons to withstand impact, but as will be discussed later adaptive constructions provide opportunities for smaller or micro-tips having even smaller diameters without departing from the scope and spirit of the present invention.

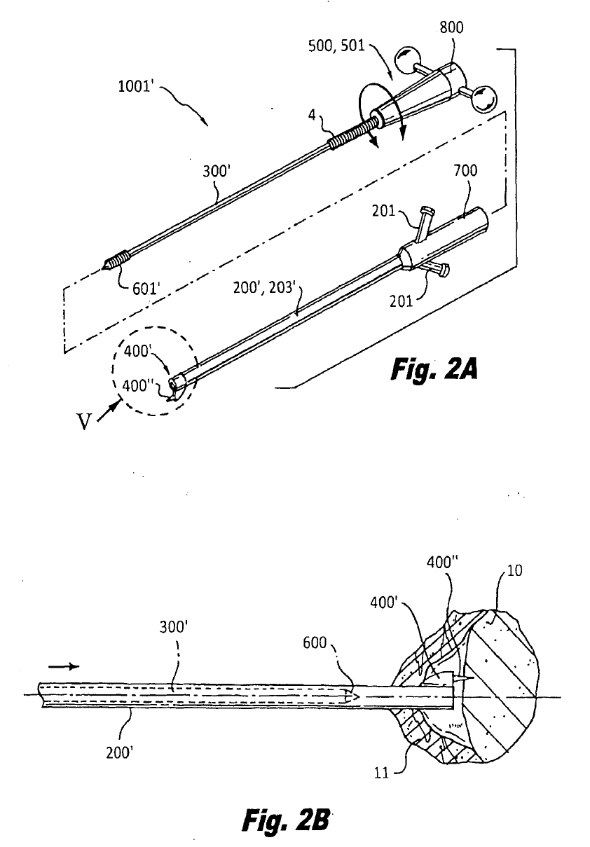

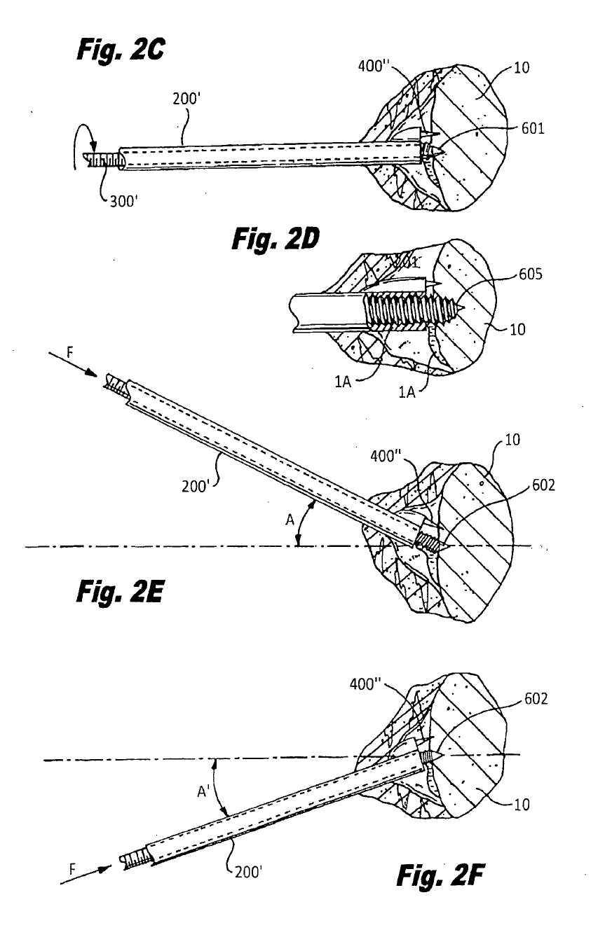

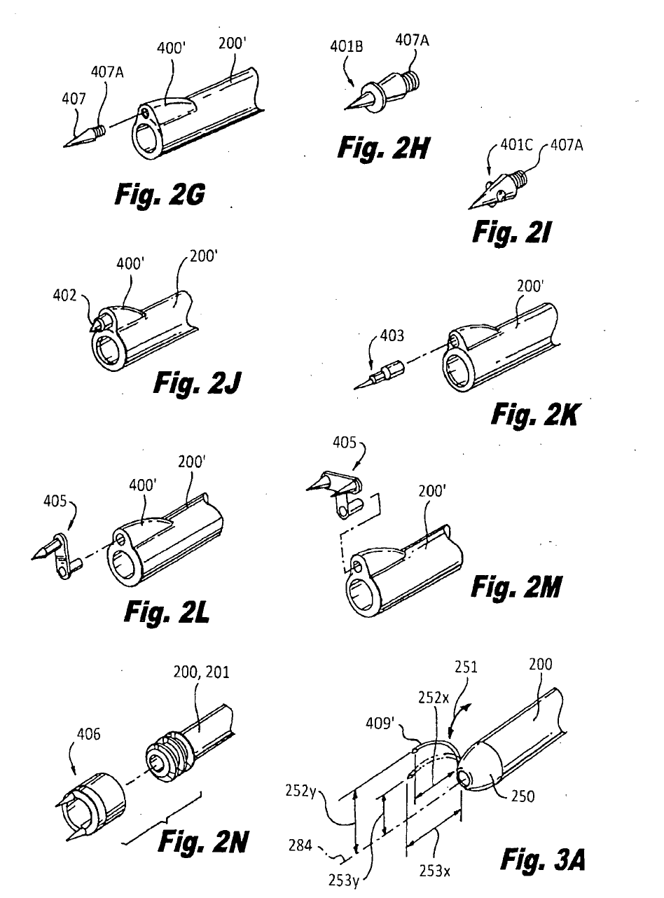

Alternatively, trocar 301 is shown with a tip end 601 having outwardly projecting rings or threads, optionally leaving a smooth cone tip (as shown) or having a threaded cone tip (See FIG. 2D and tip end 605). In FIG. 10, a trocar 302 may include a tip end 601′ having an alternative slant angle or slant tip feature 602 having optional angles at for examples, 5, 10, 15, or 20 degrees from the axis or more depending upon a surgeons or manufacturer's desire.

In FIG. 1Q, a trocar 303 is shown having a hollow tip end 603 ringed with optional saw teeth for bone cutting or optional abrading teeth members for mechanical debraiding at low speed using, for example T-handle system 800 earlier discussed.

In FIG. 1P, a trocar 304 includes (optionally) a dual end having an outward threaded profile 304A with an inner core region 304B with an annular cutting ring member (as shown) and a forwardly projecting narrow pin member or needle trocar portion 305.

Referring now to FIG. 2A, a third cannulated microfracture system 1001′ includes a trocar 300′ having respective contact tip ends 601′ on a proximal end thereof (shown here as preferably co-axially located but this is not required), threadably adjustable striker system 500 has a threadably-adjustable striker member 501 on a distal end thereof and a handle member or grip/striking assembly 800, similar to the discussion in FIGS. 1A, 1B.

A cannula 200′ includes an axis set or pick end 400′ on a proximal end and a sheath end 700 on a distal end thereof. Pick end member 400′ includes, in this embodiment, and a single pick point 400″. Sheath end 700 includes optional ports 201, 201 adaptive to threadably accept a Luer Lock Tip device (an example is produced by Terumo Medical Corp, Elkton, Md. 21921) (not shown), or optionally a fluid flow member 1 and vacuum member 2, or any other common surgical supply/suction or aid system used conventionally during orthopedic surgery.

Referring to FIG. 2B a close-up view of the embodiment of FIG. 2A is positioned accordingly to the method noted in FIGS. 1D-1F with skin 11 snugly about an outer surface of cannula 300′ for sealing with pick end 400′ having a single pick 400″, securely positioning the proximal end of canula 200′. In this assembly, a user may readily vary the positioning and depth control of trocar 300′, in the manner noted above by varying the threaded position or set position of striker 500 along threaded region 4. As will be appreciated, where solely strilcing is desired, it is possible to remove pin 803 from handle set 800 to expose the striking surface, which may be in any suitable form for preferred striking without departing from the scope and spirit of the present invention.

Referring now to FIGS. 2C through 2F, a trocar 300′ sliding within cannula 200′ is positioned relative to point 400″ and force applied thereby allowing point set 601 to penetrate the bone causing a first microfracture. Referring now to FIG. 2D, it is alternatively noted, that a trocar tip end 605 having a threaded end may be both driven without twisting and threaded/screwed into bone 10, depending upon user preference.

In FIGS. 2E-2F, it is illustrated that pick point 401′ on cannula 200′ allows a user to pivot system 1001′ an optional angle A, A′ relative to an initial pick point position CL on bone 10 so as to allow a user to control a direction of microfracture relative to an initial centerline CL.

In FIGS. 2G through 2M generally, a wide variety of set point geometries are possible without departing from the scope of the present invention, each understandable by one of skill in the art based upon the disclosure herein and the supporting images. It will be noted, that while many pock points may be shown as removable and selectable, fixably securing these differing geometries is within the scope of the present invention.

Referring now to FIG. 32G, cannula 200′ is provided with a wide variety of tips, including axis set point geometry 400′ having a single long pick point 407 removably joinable with set geometry 400′ by means of threads 407A. In FIGS. 2H and 2I it will be understood by those of skill in the art that long pick point 407 may additionally include depth stop mechanisms such as an annular ring 401B or a series of outwardly bulging members 401C each respectively serving as a stress concentrator and stress raiser when urged into a bone surface so as to minimize or prohibit unintended bone penetration beyond a desired depth. Similarly, the pick points in FIG. 2G-2I are threadably joined to set point geometry 400 as an optional feature of the invention although fixable connections may be preferred by manufacturers.

Referring now to cannula 200′ which includes a set point geometry 400′ now joined with a bull nose pick point 402, providing a high-contact angle with bone so as to minimize detrimental bone penetration without significant pressure. Due to the wide contact angle (greater than say 90 degrees) or any of the other adaptations herein, a user may gain the benefit of a pick point without the negatives of unintended bone penetration.

Referring now to FIG. 2K, an alternative stepped pick point 403 is secured to cannula 200, either removably or fixably, depending upon manufacturer need. As noted above, stepped pick point 403 provides a series of wider diameters growing from an initial cone-shaped pint, so that for example, at a first force amount F, the first cone-shaped point penetrates the bone, but requires a doubling of the first force (F2) to push past the next step, and so forth. As a consequence, a user may readily appreciate that some bone is brittle or damaged and may only require a light contact to positionably secure the end of cannula 200 to a bone location.

Referring now to FIG. 2L, a similarly adaptive end 404 is provided on cannula 200′ so as to allow tip end 404 to project away from the end of cannula 200′ for a distant securing location and thereby allow a greater range of positioning for revascularization.

Referring similarly now to FIG. 2M, similarly to the embodiment noted in FIG. 2L, a tip end 405 of cannula 200′ includes both a lateral extension member and two projecting pick points, as shown (see FIG. 2L for dual points). As a consequence of the present design, a user may “rock” cannula 200′ between the two points of a two-pointed version of 405 to gain additional freedom of use.

Referring now to FIG. 2N, a cannula has a replacement end system 201 threadably joining an annular dual pick member 406 threadably fixed to end system 201 on cannula 200 so as to bring the benefit of both a system to allow replacement of cannula tip ends but also the benefit of “rocking” or shifting cannula 200 between either point so as to move it's position a repeatable and reliably predictable distance from a first location.

As will be noted from studying FIG. 3A, an alternative tip end construction is provided for cannula 200 with features that ease use in certain circumstances. As shown, a curved or smoothly rounded end 250 or bullet end 250 is provided for easing through flesh layer 11 upon initial insertion. Similarly a flexible pick point 409 shaped as a thin-finger projecting proximate rounded end 250.

As shown pick point member 409′ may be constructed at a variety of positions and of a variety of shapable materials (such as memory metal, or plastically deformable metal), within a kit for example, and replacably or fixably mounted on the end of cannula 200 in a threaded or other manner similar to that noted in FIG. 2N. Here, a first angle for pick point member 409 provides a greater spacing 252Y from a cannula axis 284, but is correspondingly closer at length 252X. Similarly, where pick point member 409 is positioned closer to centerline 284, the tip end projects further at length 253X but provides a correspondingly narrower extension at length 253Y. As will be understood from those of skill in the art having viewed FIG. 3A, a wide variety of cannula axis sets or pick points may be adaptively employed for patient benefit without departing from the scope and spirit of the present invention.

As a further modification of the present discussion, it is proposed that pick point member 409 may be alternatively constructed from a memory-metal—namely a metallurgical allow that is responsive to a thermal inducement to change it's position relative to an initial shifted position. As a consequence, the present disclosure suggests the use of a memory metal for constructing point member 409 thereby allowing a user to merely bend point member 409 into closer alignment with axial center 284 at a “room temperature” of less than approximately 85° F. to allow easier insertion through an opening in the skin. It is further suggested that upon entry of the body at approximately 98° F., the temperature change will cause point member 409 to return to its original position allowing convenient insertion and use.

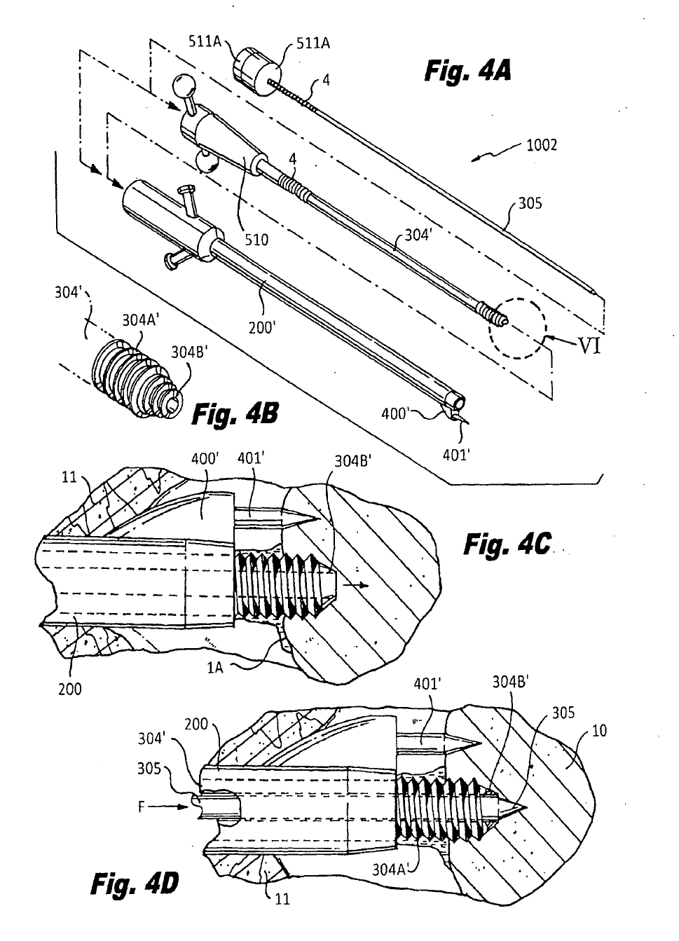

Referring now to FIGS. 4A through 4D, an alternative revascularization assembly, system, or kit 1002 is provided with cannula 200′ having an adaptive axis set or end 400′ with a prick or point 401′ as discussed above, although any of the alternatively disclosed axis set ends or pick points may be employed without departing from the scope and spirit of the present invention. An adaptive trocar 304′ includes a continuous threaded outer band 304A′ and a formed cutting ring 304B′ on a proximal end and a striker 510 on a distal end thereof as shown and discussed earlier. As noted, the depth adjustment system is similarly provided herein, as shown. Trocar 304′ is particularly formed with a hollow channel, in a manner similar to cannula 200′, so as to allow optional insertion of a further extending needle trocar 305 having a striking end 511 there through. While striking end 511, has a threadably adjustable and positionable member 511A and respective adjustment threads 4 the operation will be similarly recognized as similar to adjusting the earlier adjustable penetration trocar by those of skill in the art.

As shown particularly in FIGS. 4B and 4D, during use, a physician or surgeon may position first trocar 304′ through cannula 200′ and either drive or twist and screw end 304A′ into bone 10 below flesh layer 11. Thereafter, first trocar 304′ may be removed for repositioning to promote revascularization or alternatively left in place. However, in yet a further alternative, where the physician determines sufficient penetration has not yet been achieved, needle trocar 305 may be inserted into second cannula/trocar member 304′ and thereby further penetrate bone 10 (see FIG. 4D). Similarly in the alternative as determined by a qualified user, system 1002 may be operated with only cannula 200′ and needle trocar 305 (without trocar 304′) so as to allow substantial operating space between an outer perimeter of needle trocar 305 and the inner surface of cannula 200 so as to allow rapid flush/vacuum/debris removal cycles via ports 201, 201 or for other medical purposes as are readily apparent to those of skill in the art.

As can be visualized herein, element 304′ serves both as a cannula and as a trocar depending upon a user's desire and patient desires. Similarly, the above-discussed depth or drive stop system is readily adapted employing threads 4 so as to allow adjustment of striker heads 510, 511 relative to their respective distal contact ends. In the present embodiment in FIG. 4A, it should be understood by those of skill in the art that two depth or drive stop systems are provided on respective trocar 304′ and 305. As will be further appreciated the shape of striker heads 501, 511 is not controlling, and alternative shaped striker head constructions may be employed without departing from the scope of the present invention. For example, a triangular or rectangular or rectilinear shaped striking head may be employed.

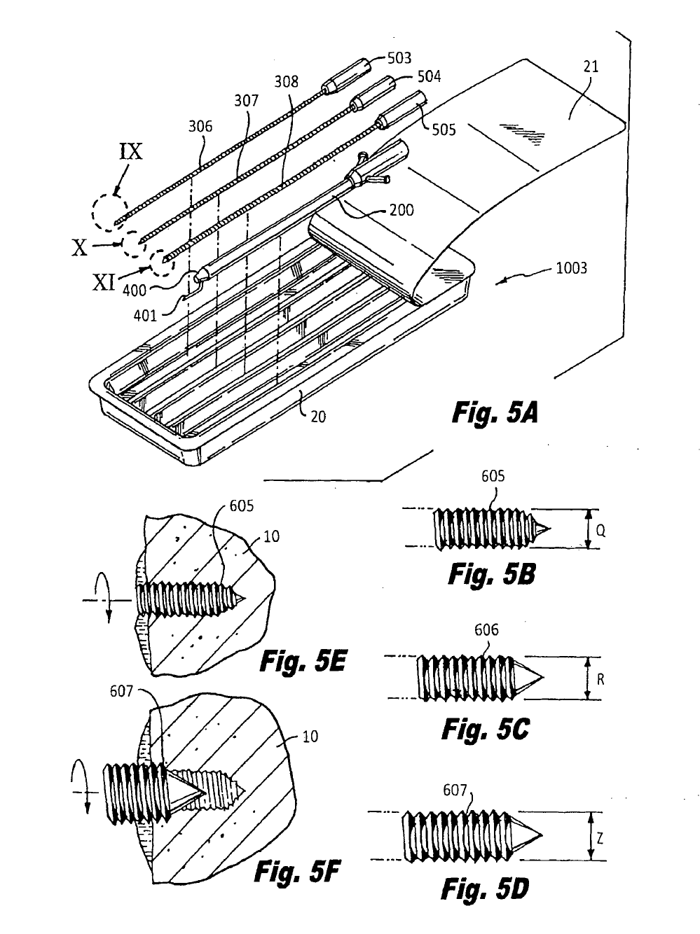

Referring now to FIG. 5A, where a system, kit or set 1003 of alternatively formed items is provided in a packaging member 20 having a readily removable cover top 21. It is envisioned, that system 1003 may be readily pre-packaged in a sterile environment before being transported to a use arena, whereupon a user may simply peel-off layer 21, which may be formed from an opaque, transparent, or translucent materials as desired by a user. Layer 21 may containing identifiable instructions or other images or words on an outer surface thereof.

While any cannula noted herein may be readily so packaged, FIG. 5A illustrates the enclosure of cannula 200, as well as three alternative style trocar members, respectively 306, 307, and 308 having respective striking ends 503, 504, and 506, as will be discussed in detail. As will be apparent to one of skill in the art, kit or system 1003 may be pre-assembled for convenience and include any of the elements discussed herein. Similarly, it will be recognized that tray 20 for kit or system 1003 may be easily resized or re-organized according to a user's need without escaping the scope of the present invention disclosed herein. Thus for example, two cannulas may be provided with eight trocars and rotation head assembly 800 without departing from the spirit and scope of the present invention.

Referring now to FIGS. 5B, 5C, and 5D differing tip ends 605, 606, and 607 are provided on respective trocars 306, 607, and 308 as noted. Tip ends 605, 607, and 607 vary by outer diameters respectively Q, R, and Z. Similarly, it will be noted that tip end 605 is a threaded end, while tip end 606 is a narrow ring end, and tip end 607 is a wide ring tip end.

Referring now to FIGS. 5E and 5F, it is envisioned that trocar 306 having threaded tip end 605 is threaded into bone 10 provided a threaded cavity as a form of pilot hole for later use. Trocar 306 is thereafter reverse threaded out of the pilot hole allowing entry of trocar 307 having tip end 606. As a result of the prior-created threaded pilot hole, trocar 307 ready follows the same path in bone 10 and similarly expands the opening to aid surgical healing. As a consequence, while any operative manner may be employed from the related embodiments, the present embodiment provides a possible pre-assembled kit structure for use in a critical surgical environment.

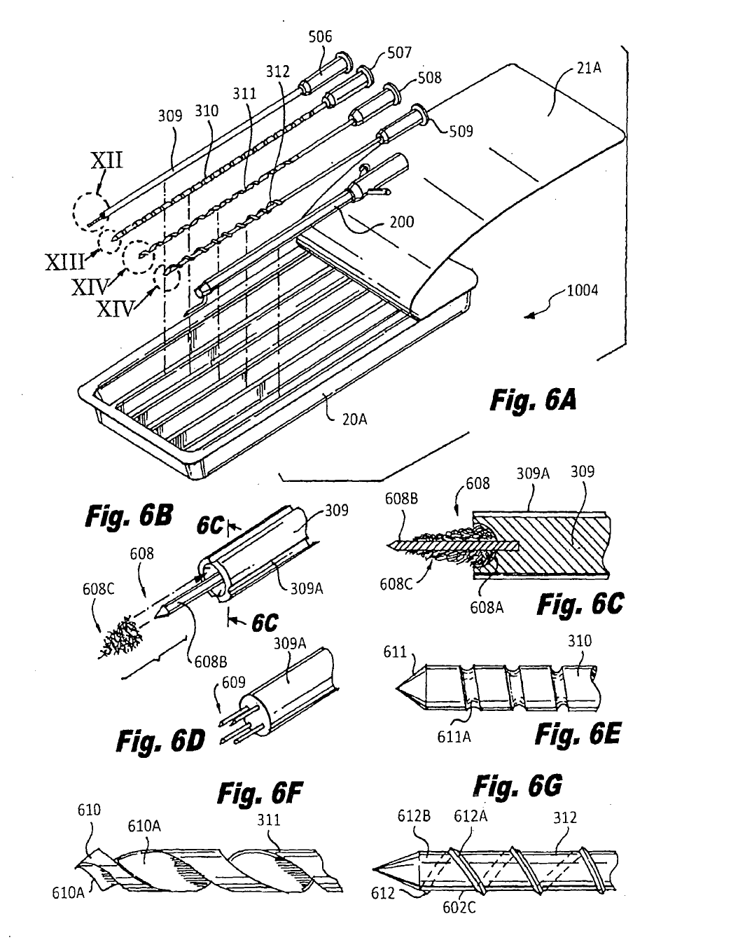

Referring now to FIG. 6A, an alternatively adapted kit or system 1004 is provided having a tray 20A with a peel-away cover 21A operating similarly in the manner noted above. As was earlier the case, cannula 200 having a tip end is provided as a representative example but those of skill in the art will readily recognize that alternatively constructed cannula and selectable tip ends as discussed herein may be substituted without departing from the scope and spirit of the present invention.

As further alternatives to the above-noted trocar constructions, a plurality of differently constructed trocars 309, 610, 311, and 312 are providing having respective striker ends 506, 507, 508, and 509 opposing respective contact ends 608, 610, 611 and 612, as will be discussed.

Referring now to FIGS. 6B and 6C, trocar end 608 is provided with an outer shaft, in this case having transverse external channels 309A formed about an outer periphery to allow transport of fluid 1 and removal of the same and debris during use.

A concave region or cup 608A surrounds a spiked tip end or pin 608B that provides a supportive contact member for transporting a biological material 608C, such as growth medium to a desired location. As a consequence, it is envisioned that the present embodiment operates as a transport system for enabling accurate positioning of growth medium within a previously prepared microfracture location. An alternative construction of this system, an adapted micro-pin end 609 contains a plurality of extending tiny-sized pin members thereby allowing a user to pack bone growth medium or another treatment medium or a treatment transport medium such as a dissolvable sponge about tiny pins on micro pin ends 609. As will be readily understood by those of skill in the art, when employing the trocar end embodiments in FIGS. 6B and 6D, a microfracture is preferably made by an earlier-applied trocar, which is then removed from cannula 200 and replaced with trocar 309 carrying bone growth or vascular growth medium.

Referring now to FIGS. 6E, 6F, and 6G, adaptive trocar tip ends are discussed for aid during microfracture operations. As noted in FIG. 6E, tip end 611, and in this case the shaft of trocar 310, contains a helical channel 611A about an outer periphery to aid in transport. An alternative construction noted in FIG. 6F provides for a multi-flute design for tip 610, providing opposing flutes 610A, 610A for similar reasons to those noted above. Finally, as noted in FIG. 6G, an outer spiral member 612A on tip 612, provides two transport channels 612B, 612C. In each of the examples noted above a mechanism and design to aid in fluid flow and removal of debris mechanism is provided so that those of skill in the art may recognize that the present system is readily adapted to changing surgical requirements. It is envisioned, that a surgical user may now select with precision a trocar tip end for a particular surgical need without departing from the scope of the present discussion.

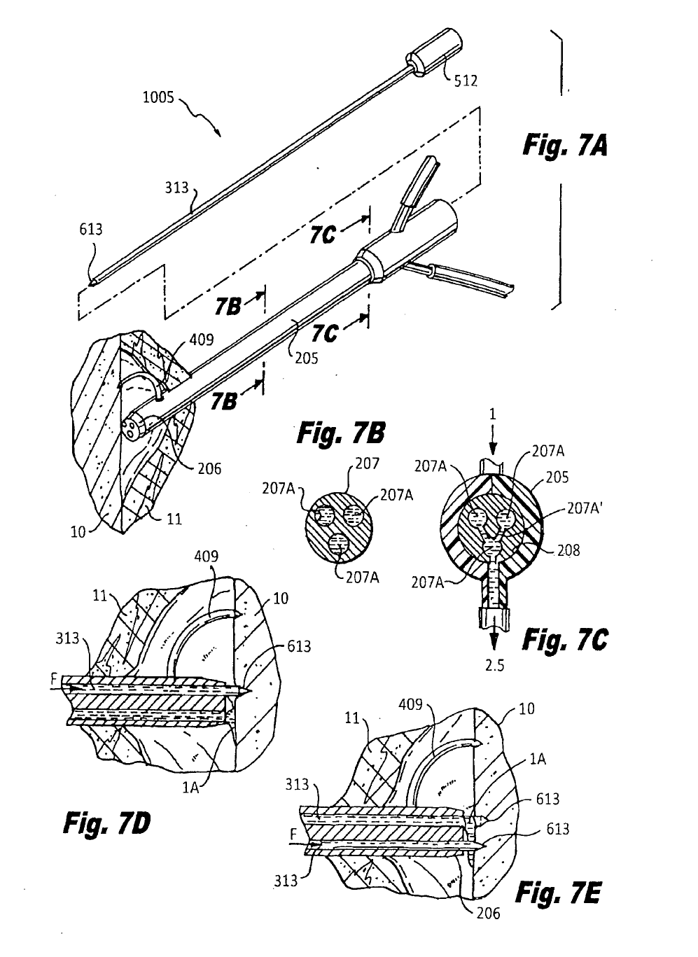

Referring now to FIGS. 7A through 7E, a kit, system, or assembly 1005, contains as desired, a cannula 205 having a pick set of pick point 409 for example. At least one micro-diameter trocar 313 is provided with a striker end 512 and a micro tip end 613. Cannula 205 contains an internal division chamber 207 containing a plurality of passages 207A, inter connected by internal connections 207A′ (for inter-passage fluid flow) for slidably receiving and guiding one or more micro-diameter trocars 313 as required. It is similarly envisioned, that via ports 201, fluid flow 1 and suction 2 may readily fill, for example two of three passages 207A so as to flush debris from bone 10. As is depicted, after cannula 205 is positioned with pick point 409 in a desired location, a user may employ sufficient trocars 313 to microfracture the bone surface in a desired manner. Where more than one micro-diameter trocar 313 is employed, threadably adjustable striker ends 512 may be shaped as, for example, a triangle/pie-shape, to thereby allow the use of all three micro-diameter trocars 313. As will be obvious to those of skill in the art, the present construction allows the generation of precise, and secure micro-fractures without the need to insert multiple cannulas in a local.

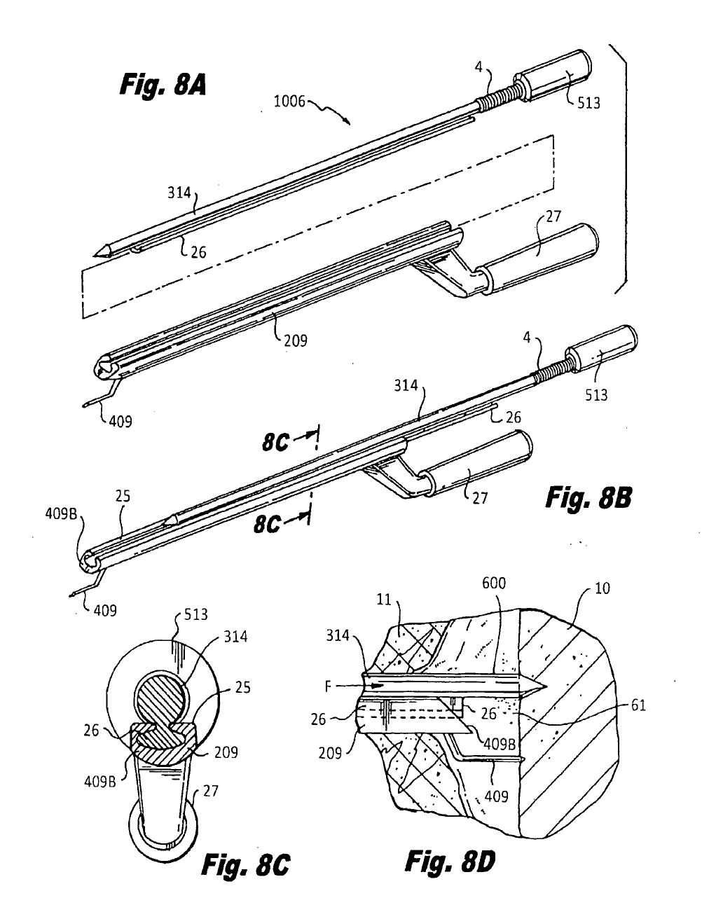

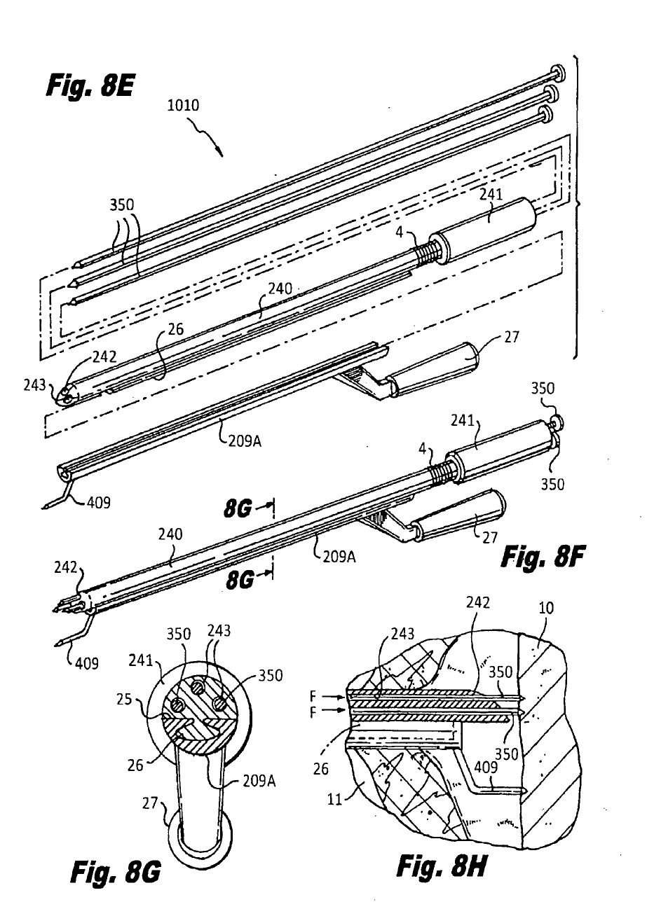

Referring now to FIGS. 8A through 8D, an alternative system, kit, or assembly 1006 is provided and employs a re-designed open-channel cannula member 209 having an off-set guiding handle 27 on a first end, and pick point 409 on the distal end thereof for positioning. A smoothly sloped end 409B operates to guide insertion through a skin opening. A trocar 314 is provided having a striker end 513 adjustable via a strike adjustment feature 4, discussed earlier. A bottom key 26 member projects from one side of trocar 314 and is slidably guided in a corresponding key channel 25 in cannula 209. In this manner, cannula 209 remains operative as a slidable guide for striking bone 10 with a tip end 600 to generate bone debris 611.

It operating system, kit, or assembly 1006 those of skill in the art will recognize the detail that striker end 513 is threadably adjustable via threads 4 along a length direction of trocar 314, while striker end 513 is of a sufficiently large diameter to contact an end of key channel 25 proximate handle 27 so as to thereby prevent further penetration, the sum construction being recognizable as depth control or penetration limitation system.

Referring now to FIGS. 8E through 8H, another alternative system, kit, or assembly 1010 is provided and employs a re-designed open channel cannula 209A having pick point 409 at a proximate end and handle 27 at a distal end. A combination trocar/cannula 240 is provided having a guiding key 26 for slidably engaging a guiding channel 25, as shown.

An end of trocar/cannula 240 distant a striking member 241 is a smoothly sloped entry zone 242, provided to ease passage through skin layer 11, and if necessary, serve as a bone-contact microfracture end. In a manner noted above, an inner portion of trocar/cannula 240 contains passages 243 for containing one or more micro-trocars 350. As was noted early, threaded range 4 allows for pivotable adjustment of strike end 241 relative to an overall length, so that upon contacting an end of channel 25, the end of handle member 241 functions as a depth stop control means. Of course, micro-trocars 350 may be similarly inserted via contact end 241 so as to create controllable micro-fractures in bone 10.

In view of treatment systems 1006 and 1010, the present invention envisions the use of cannulas 209, 209A in combination with other surgical tools, as long as each adaptive surgical tool may be slidably adapted for use along the channel so as to enter skin layer 11 smoothly and controllably without guessing. This adaptation may be of critical importance where additional surgical requirements urge the inclusion of imaging tools, sampling tools, and other testing tools all benefiting from the security provided by set or pick points 409 and the guidance provided by the above-described channel-slide construction.

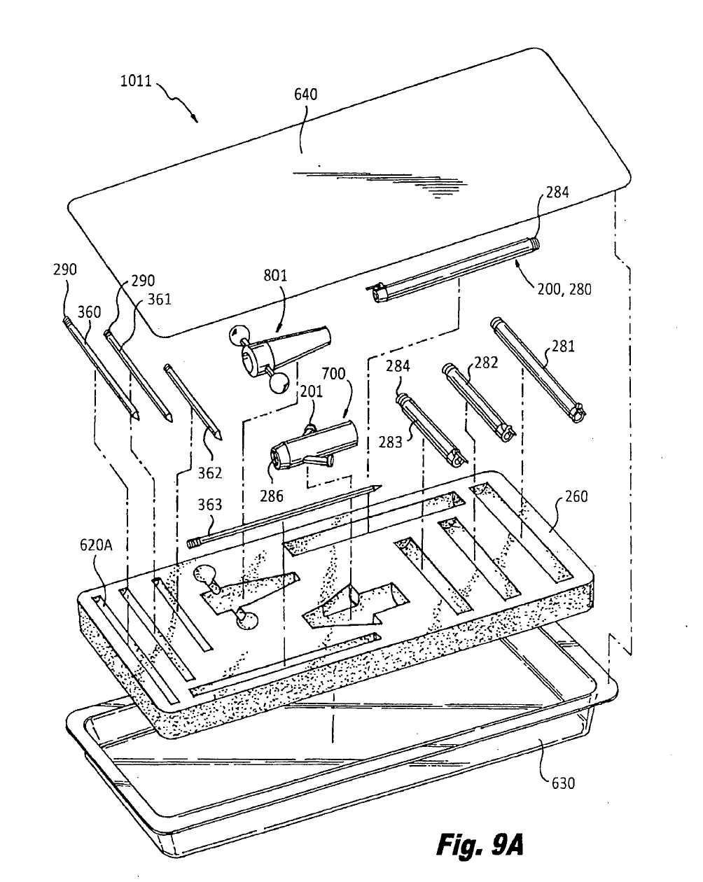

Referring now to FIG. 9A, an assembly kit system 1011 is provided with a carrying tray 630, a supportive foam inner member 620 and a peel-back cover 640 that is hermetically sealed to enclose kit 1011 between manufacture and use.

As noted, inner member 620 contains a plurality of pocket recesses 620A shaped to securely receive and stabilize respective items of the kit.

Within the recesses in inner member 620 are contained a break-down or substitutable series of components related to those described above, as will be discussed. As shown are a cannula sleeve member 200 having a threaded connection end 284 for threadably engaging a sheath end 700 having corresponding female receiving threads 286 at an end thereof and respective ports 201. In combination, cannula member 200 and sheath end 700 form the cannula element noted above; however the present kit also provides replacement cannula ends 283, 282, and 281 each having different respective lengths. For example, the present kit may contain cannulas having a lengths of 6, 8, 10, and 12 inches, although alternative lengths are readily envisioned without departing from the scope and spirit of the present invention. Each replacement cannula 280, 281, 282, and 283 may be readily selected according to a user's preference or surgical need, and each may contain a threadably removable set point constructed in a manner noted above. Similarly, a plurality of trocars 363, 362, 361, and 360 having corresponding lengths of 6, 8, 19, and 12 inches are provided, each with a respective threaded ends 290 that threadably engage striker head assemblies 801 to provide length adjustment.

As will be apparent to those of skill in the art having reviewed the disclosure herein, the length adjustment means allows adjustment of a penetration depth between a maximum and minimum of an adjustment range. For example, a user needing a penetration depth of ½ inch may select a 6-inch length cannula assembly and a 6-length trocar assembly, and loosen the striking end ½ inch so as to allow a user to drive the same to the desired depth.

In view of the alternative constructs discussed above, it is proposed that those of skill in the art of surgical instrument design will readily recognize the ready adaptation to need the present system provides.

Alternatives to the present include, but are not limited to the alternatives noted below. For trocars (300 series elements) a wide variety was noted, including those of fixed lengths, and selectable lengths having diameters of, for example 1.2 mm, 2.0 mm, 2.5 mm, 3.0 mm, 3.5 mm, 4.0 mm etc and down as small as approximately an 18 gauge needle. A wide series of trocar tips was also provided, and these include various smooth ends, threaded ends, helical ends, micro-prong ends, fixed angles, flat ended trocars (for packing bone growth medium); and concave tip ends for transporting and placing bone growth medium.

A wide variety of sheaths for the cannulas was also noted, and included variants to accept a lure-lock device, vacuum application, and fluid flows as well as other items such as imaging systems. Cannulas similarly are provided with a wide range of constructions, from tubular, to multi-exit constructions, to adaptive dove-tail type slot and groove constructions that will allow ready tool insertion into a skin opening. Similarly axis set points were proved in wide variations from those with short and long prick ends, curved prick ends, angularized “hockey-stick” type ends, depth stop ends, memory metal pick ends, dual tip ends, replacement tip ends, and wide angle ends among others.

Similarly, it will be recognized that the present invention teaches adaptation to reach surgical solutions. For example, cannulas 209, 209A do not include a sheath member 700 as noted in the opening discussion so that the present system teaches the need for ready adaptation for surgical success without requiring strict adherence to the depicted embodiments.

Also provided were a variety of assistive tools such as handle attachments to a striking end for hand-twisting and removal, ready kit packaging for transport and secure storage, and provision of a wider made-to-request system requirement so that a user may construct the systems herein at a desired length from a grouping of differently shaped parts (See for example FIG. 9A).

Additionally, it should be understood herein, that the use of the phrase trocar shall be interpreted broadly to cover generally sharp ended surgical instruments employed for applying force to a human-body element, without inferring outside limitations requiring the penetration of skin or use with flexible cannulas. Similarly, it will be understood herein, that the use of the phrase cannula or canula (both are correct spellings historically used), sheath or guide rail or guide shall be interpreted very broadly to mean a surgical device that guides, supports, aims, or is otherwise used with a trocar as described herein, without any outside limitation. Thus for example, cannula 200 (FIG. 1A) shall be understood to represent the same instrument as guiding cannula 209 (FIG. 8A) despite their differing appearance and construction.

In the claims, means- or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

What is claimed is:

1. A surgical system for treating a patient involving microfracture of an external bone surface during a use, comprising:
a canula system;
a trocar system having a proximate tip end and a distal striking end;
means for guidably receiving said proximate tip end of said trocar system along a length of said canula system from a sheathed end to a distal set point end, whereby said means for guidably receiving enables a guiding of said tip end of said trocar system proximate said external bone surface of said patient during said use; and
at least one set point means on said canula system for extending from said canula system proximate said distal set point end opposite said trocar receiving sheathed end thereof and for engaging said external bone surface during said use; whereby said surgical system enables secure positioning of said of said tip end of said trocar system relative to said external bone surface.

2. A surgical system, for treating a patient according to claim 1, further comprising:
means for adjusting a penetration depth of said trocar tip end into said bone surface.

3. A surgical system, for treating a patient according to claim 2, wherein:
said at least set point means on said proximate end of said cannula system is at least one selected from a group consisting of at least one of:
a smooth set point, a conical set point, a point including means for minimizing a bone penetration depth, a removable tip end set point, a non-removable tip end, a stepped-penetrating tip end, a flexible tip end, a multiple point end, an off-angle tip end, a memory metal tip end, and a smooth arch tip end.

4. A surgical system, for treating a patient, according to claim 1, wherein:
said proximate tip end of said trocar system is selected from a group consisting of at least one of: a pointed microfracture end, a non-pointed microfracture end, a threaded end, an internally fluted helix end, a fixed-angle end, an externally fluted end, a spiral ringed end, a channeled end, a flat-packing to end, a biological inducing end, a medium-transport end, a hole-cutting end, and a bone surface perturbing end.

5. A surgical system, for treating a patient, according to claim 1, further comprising:
a trocar system having at least one through passage; and
at least one additional micro-trocar member passable through said through passage.

6. A surgical system, for treating a patient, according to claim 1, wherein:
said means for guidably receiving said proximate tip end of said trocar system along said length of said canula system further comprises at least one of the following:
means for slidably guiding said trocar system through an axially related through-opening along a length of said canula system, whereby a portion of said trocar system is internal of said canula system during said use, and means for slidably guiding said trocar system along an axially related direction on an external length portion of said canula system during said use.

7. A surgical system, for treating a patient, according to claim 2, wherein:
said means for adjusting a penetration depth of said trocar tip end into said bone surface, further comprises:
a striking member on said distal striking end of said trocar system;
means for adjustably positioning said striking member at a plurality of locations relative to an extending body shaft of said trocar system, and
said means for adjustably positioning said striking member adjustably controlling said length of said trocar system that is guidably received by said means for guidably receiving said trocar system along said length of said canula system.

8. A surgical system, for treating a patient, according to claim 7, wherein:
said means for adjustably positioning said striking member further comprises:
an adjustable sheathed end member on said sheathed end of said canula system;
means for threadably positioning said adjustable sheathed end member relative to said length of said canula system, whereby a penetrating length of said proximate tip end of said trocar system may further penetrate said external bone surface during said use.

9. A surgical system, for treating a patient, according to claim 2, wherein:
said means for adjusting a penetration depth of said trocar tip end into said bone surface, further comprises:
a striking member on said distal striking end of said trocar system;
means for adjustably positioning said striking member at a plurality of locations relative to an extending body shaft of said trocar system,
an adjustable sheathed end member on said sheathed end of said canula system; and
means for threadably positioning said adjustable sheathed end member relative to said length of said canula system, whereby a penetrating length of said proximate tip end of said trocar system may further penetrate said external bone surface during said use.

10. A surgical kit system, comprising:
a trocar system;
a cannula system for receivably accepting said trocar system;
pick point end on said cannula system enabling secure position on an external patient bone surface;
means for adjusting a movement of said trocar system relative to said cannula system;
striking means on an end of said trocar system opposite a bone contact end;
sheath means in said cannula system for guiding said trocar system and for providing at least one access port to said cannula system; and
openable kit storage means for storing elements of said surgical kit system prior to a use thereof.

11. A surgical kit assembly, comprising
a kit-holding member;
a trocar system;
a cannula system for slidably accepting said trocar system;
said kit-holding member including a plurality of holding locations for receiving at least elements of both said trocar system and said cannula system; and
means for removably sealing said kit-holding member to provide a user-digital access to respective said plurality of holding locations.

12. A method for treating bone, comprising the steps of:
providing a trocar system having a proximate bone-contacting end and a distal striking end opposite said bone-contacting end;
providing a cannula system for receivably guiding at least said bone-contacting portion of said trocar system into an external bone contact;
positioning a pick point on a proximate end of said cannula system for contacting said bone, whereby said pick point enables ready positioning of said bone-contacting end of said trocar system; and
striking said distal striking end of said trocar system so that said step of striking drives a least a portion of said bone-contacting end into a contact with said bone causing a micro-fracturing thereof.

13. A surgical system for treating a patient involving microfracture of an external bone surface during a use, comprising:
a canula system member having a sheathed end opposite a set point end;
a trocar system having a proximate tip end opposite a distal striking end;
means for guidably receiving said proximate tip end of said trocar system along a length of said canula system, and
at least one axial set point system means on said set point end extending opposite said trocar receiving sheathed end thereof for engaging said external bone surface during said use; whereby said surgical system enables secure positioning of said of said tip end of said trocar system relative to said external bone surface.

14. A surgical system, according to claim 14, wherein:
said means for guidably receiving enables a guiding of said tip end of said trocar system proximate said external bone surface of said patient during said use.

15. An adjustable trocar system, comprising:
a trocar surgical microfracture member having a microfracture tip end and a distal striking end;
means for threadably adjusting a distance between said microfracture tip end and said distal striking end; and
said microfracture tip end is selected from a group consisting of at least one of a pointed microfracture end, a non-pointed microfracture end, a threaded end, an internally fluted helix end, a fixed-angle end, an externally fluted end, a spiral ringed end, a channeled end, a flat-packing end, a biological inducing end, a medium-transport end, a hole-cutting end, and a bone surface perturbing end.

16. An adjustable trocar system, according to claim 15, further comprising:
a removable hand gripping system means for releasably engaging said distal striking end of said trocar surgical microfracture member.

17. An adjustable trocar system, according to claim 16, further comprising:
at least one extending hand-grip handle projecting from said removable hand gripping system, whereby during a use of said adjustable trocar system, a user may digitally grasp said extending hand-grip handle and manipulate said trocar surgical microfracture member during a use thereof.

18. An adjustable canula system, comprising:
a canula system member having a sheathed end opposite a set point end, each positioned relative to a canula system axis;
at least one set point means on said proximate end of said cannula system;
said at least one set point means including a contact-tip end extending coaxially to said canula system axis; and
said at least one set point means being selected from a group consisting of at least one of: a smooth set point, a conical set point, a point including means for minimizing a bone penetration depth, a removable tip end set point, a non-removable tip end, a stepped-penetrating tip end, a flexible tip end, a multiple point end, an off-angle tip end, a memory-metal tip end, and a smooth arch tip end.

19. A surgical system for treating a patient involving microfracture of an external bone surface during a use, comprising:
a canula system;
a trocar system having a proximate tip end and a distal striking end;
means for guidably receiving said proximate tip end of said trocar system along a length of said canula system from a sheathed end to a distal set point end, whereby said means for guidably receiving enables a guiding of said tip end of said trocar system proximate said external bone surface of said patient during said use;
at least one set point means on said canula system for extending from said canula system proximate said distal set point end opposite said trocar receiving sheathed end thereof and for engaging said external bone surface during said use; whereby said surgical system enables secure positioning of said of said tip end of said trocar system relative to said external bone surface;
and at least one of a fluid flow port, a vacuum port, and a medical product-inducing port on said canula system, whereby said surgical system enables an enhanced treatment of said patient.
